(19) United States
(12) Patent Application Publication
(10) Pub. No.: US 2024/0206827 A1
(43) Pub. Date: Jun. 27, 2024

(54) TABLE PANEL ASSEMBLY, EXAMINATION TABLE, AND X-RAY IMAGING SYSTEM

(71) Applicant: GE Precision Healthcare LLC, Waukesha, WI (US)

(72) Inventors: Fusheng Li, Beijing (CN); Yuqing Li, Beijing (CN); Peng Qi, Beijing (CN); Yigang Xia, Beijing (CN); Youbao Cao, Beijing (CN)

(21) Appl. No.: 18/533,697

(22) Filed: Dec. 8, 2023

(30) Foreign Application Priority Data
Dec. 27, 2022 (CN) 202211683428.1

(51) Int. Cl. A61B 6/04; A61B 6/00

(52) U.S. Cl. CPC A61B 6/0407; A61B 6/42

(57) ABSTRACT

The present application provides a table panel assembly, an examination table, and an X-ray imaging system. The table panel assembly is arranged on a base of the examination table and is used for bearing an object under examination. The table panel assembly includes a support frame and a table panel. The support frame is mounted on the base. The table panel is mounted on the support frame. The table panel is movably connected to the support frame. A rotating shaft is formed between the table panel and the support frame. The table panel can be flipped outward relative to the rotating shaft. An accommodating space is formed between the table panel and the support frame, and the accommodating space can be used for placing and moving a detector.

### CROSS REFERENCE TO RELATED APPLICATIONS

The present application claims priority and benefit of Chinese Patent Application No. 202211683428.1 filed on Dec. 27, 2022, which is incorporated herein by reference in its entirety.

### TECHNICAL FIELD

The present invention relates to medical imaging technologies, and specifically to a table panel assembly, an examination table, and an X-ray imaging system.

### BACKGROUND

In an X-ray imaging system, radiation from an X-ray source is emitted toward a subject, and the object under examination is usually a patient in a medical diagnosis application. A part of the radiation passes through the object under examination and impacts a detector, which is divided into a matrix of discrete elements (e.g., pixels). The detector elements are read to generate an output signal on the basis of the amount or intensity of radiation that impacts each pixel region. The signal can then be processed to generate a medical image that can be displayed for review, and the medical image can be displayed in a display apparatus of the X-ray imaging system.

For a lying-position scanning mode, typically, the object under examination needs to lie on an examination table in a lying position. The examination table includes a base and a table panel. The table panel is floatingly mounted on the base. The detector is mounted within the space between the table panel and the base, and the table panel can move relative to the base, so that a region of interest of the object under examination reaches the region of the detector. Since the table panel needs to move relative to the base, the space required for a scan room and/or the examination table region is also large.

In addition, for the maintenance and/or replacement of the table panel and/or other devices inside of the examination table, the table panel may need to be moved away from the base and then removed, or the table panel may need to be completely dismounted, either of which requires a large space and a complicated process.

### SUMMARY

The present invention provides a table panel assembly, an examination table, and an X-ray imaging system.

Exemplary embodiments of the present invention provide a table panel assembly. The table panel assembly is arranged on a base of an examination table and is used for bearing an object under examination. The table panel assembly comprises a support frame and a table panel. The support frame is mounted on the base. The table panel is mounted on the support frame. The table panel is movably connected to the support frame. A rotating shaft is formed between the table panel and the support frame. The table panel is capable of being flipped outward relative to the rotating shaft. An accommodating space is formed between the table panel and the support frame, and the accommodating space is capable of being used for placing and moving a detector.

The exemplary embodiments of the present invention further provide an examination table. The examination table comprises a base, a support frame, a table panel, and a detector assembly. The support frame is mounted on the base. The table panel is mounted on the support frame. The table panel is movably connected to the support frame. A rotating shaft is formed between the table panel and the support frame, and an accommodating space is formed between the table panel and the support frame. The detector assembly is mounted within the accommodating space, and the detector assembly is capable of moving relative to at least one direction among transverse and longitudinal directions of the table panel.

The exemplary embodiments of the present invention further provide an X-ray imaging system. The X-ray imaging system comprises an examination table. The examination table comprises a base, a support frame, a table panel, and a detector assembly. The support frame is mounted on the base. The table panel is mounted on the support frame. The table panel is movably connected to the support frame. A rotating shaft is formed between the table panel and the support frame, and an accommodating space is formed between the table panel and the support frame. The detector assembly is mounted within the accommodating space, and the detector assembly is capable of moving relative to at least one direction among transverse and longitudinal directions of the table panel.

Other features and aspects will become apparent from the following detailed description, drawings, and claims.

### BRIEF DESCRIPTION OF THE DRAWINGS

The present invention can be better understood by means of the description of the exemplary embodiments of the present invention in conjunction with the drawings, in which:

FIG. 1 is a schematic diagram of an X-ray imaging system according to some embodiments of the present invention;

FIG. 2 is a schematic diagram of an examination table according to some embodiments of the present invention;

FIG. 3 is a schematic diagram of a table panel assembly in a first state according to some embodiments of the present invention;

FIG. 4 is a cross-sectional view of the table panel assembly shown in FIG. 3 along A-A′;

FIG. 5 is a schematic diagram of the table panel assembly in a second state according to some embodiments of the present invention;

FIG. 6 is a schematic diagram of the table panel assembly in a third state according to some embodiments of the present invention;

FIG. 7 is a schematic diagram of a table panel assembly according to some embodiments of the present invention; and

FIG. 8 is a schematic diagram of a detector assembly according to some embodiments of the present invention.

### DETAILED DESCRIPTION

Specific embodiments of the present invention will be described below. It should be noted that in the specific description of said embodiments, for the sake of brevity and conciseness, the present description cannot describe all of the features of the actual embodiments in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first” and “second” and similar terms used in the description and claims of the patent application of the present invention do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

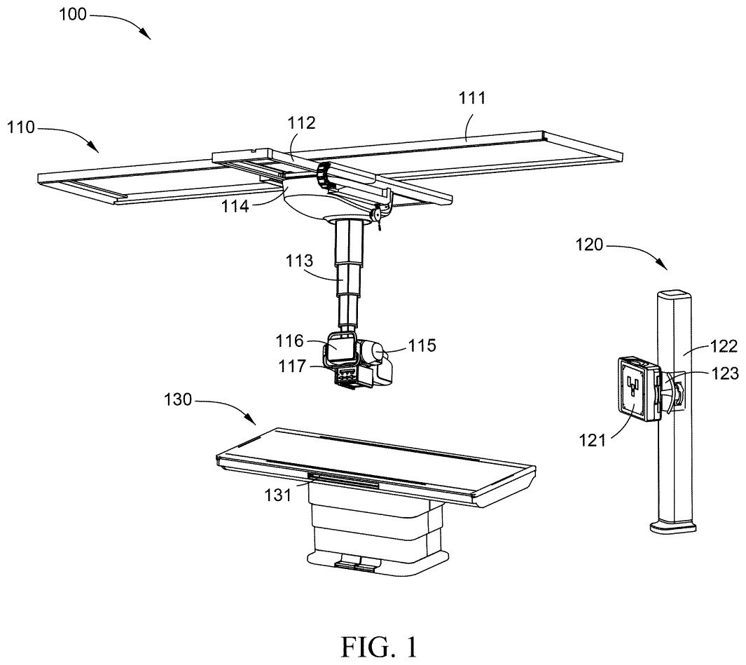

FIG. 1 shows an X-ray imaging system 100 according to some embodiments of the present invention. As shown in FIG. 1, FIG. 1 shows the X-ray imaging system 100 according to some embodiments of the present invention. As shown in FIG. 1, the X-ray imaging system 100 includes a suspension apparatus 110, a wall stand apparatus 120, and an examination table apparatus 130. The suspension apparatus 110 includes a longitudinal guide rail 111, a transverse guide rail 112, a telescopic cylinder 113, a sliding member 114 and a tube assembly 115.

For ease of description, in the present invention, the x-axis, y-axis, and z-axis are defined as the x-axis and y-axis being located in the horizontal plane and perpendicular to one another, and z-axis being perpendicular to the horizontal plane. Specifically, the direction in which the longitudinal guide rail 111 is located is defined as the x-axis, the direction in which the transverse guide rail 112 is located is defined as the y-axis direction, and the direction of extension of the telescopic cylinder 113 is defined as the z-axis direction, and the z-axis direction is the vertical direction.

The longitudinal guide rail 111 and the transverse guide rail 112 are perpendicularly arranged, wherein the longitudinal guide rail 111 is mounted on the ceiling and the transverse guide rail 112 is mounted on the longitudinal guide rail 111. The telescopic cylinder 113 is configured to carry the tube assembly 115.

The sliding member 114 is provided between the transverse guide rail 112 and the telescopic cylinder 113. The sliding assembly 114 may include components such as a rotating shaft, a motor, and a reel, etc. The motor can drive the reel to rotate around the rotating shaft, which in turn drives the telescopic cylinder 113 to move along the z-axis and/or slide relative to the transverse guide rail. The sliding member 114 is capable of sliding relative to the transverse guide rail 112, i.e., the sliding member 114 is capable of driving the telescopic cylinder 113 and/or the tube assembly 115 to move in the y-axis direction. Further, the transverse guide rail 112 can slide relative to the longitudinal guide rail 111, which in turn drives the telescopic cylinder 113 and/or the tube assembly 115 to move in the x-axis direction.

The telescopic cylinder 113 includes a plurality of cylinders having different inner diameters, and the plurality of cylinders can be sleeved, sequentially from bottom to top, in the cylinder located thereabove, thereby achieving telescoping, and the telescopic cylinder 113 can be telescopic (or movable) in the vertical direction, i.e., the telescopic cylinder 113 can drive the tube assembly to move along the z-axis direction. The lower end of the telescopic cylinder 113 is further provided with a rotating part, and the rotating part can drive the tube assembly 115 to rotate.

The tube assembly 115 includes an X-ray tube, and the X-ray tube may produce X-rays and project the X-rays towards an intended region of interest (ROI) of a patient. Specifically, the X-ray tube may be positioned adjacent to a beam limiter, and the beam limiter is used to align the X-rays with the intended region of interest of the patient. At least part of the X-rays may be attenuated by means of the patient and may be incident on a detector assembly 121/131.

The suspension apparatus 110 further includes the beam limiter 117, which is usually mounted below the X-ray tube, and the X-rays emitted by the X-ray tube irradiate the body of an object under examination by means of an opening of the beam limiter 117. The irradiation range of the X-rays, i.e., a region size of an exposure field of view (FOV), is decided by the size of the opening of the beam limiter 117. The position of the exposure field of view (FOV) on the body of the object under examination is decided by the positions of the X-ray tube and the beam limiter 117 in the transverse direction. It is well known that X-rays are harmful to the human body, and it is thus necessary to control the X-rays, so that the X-rays only irradiate a site of the object under examination that needs to be examined, i.e., the region of interest (ROI).

The suspension apparatus 110 further includes a tube control apparatus (console) 116. The tube control apparatus 116 is mounted on the tube assembly. The tube control apparatus 116 includes user interfaces such as a display screen and a control button for performing preparation work before image capture, such as patient selection, protocol selection, positioning, etc.

The movement of the suspension apparatus 110 includes the movement of the tube assembly along the x-axis, y-axis, and z-axis, as well as the rotation of the tube assembly in the horizontal plane (the axis of rotation is parallel to or overlaps with the z-axis) and in the vertical plane (the axis of rotation is parallel to the y-axis). In the above movement, a motor is usually used to drive a rotating shaft which in turn drives the corresponding components to rotate in order to achieve the corresponding movement or rotation, and the corresponding control components are normally mounted in the sliding member 114. An X-ray imaging unit further includes a motion control unit (not shown in the figure), and the motion control unit can control the above-mentioned movement of the suspension apparatus 110. Furthermore, the motion control unit can receive a control signal to control a corresponding component to move correspondingly.

The wall stand apparatus 120 includes a first detector assembly 121, a wall stand 122, and a connecting portion 123. The connecting portion 123 includes a support arm that is vertically connected in the height direction of the wall stand 122 and a rotating bracket that is mounted on the support arm, and the first detector assembly 121 is mounted on the rotating bracket. The wall stand apparatus 120 further includes a detector driving apparatus that is arranged between the rotating bracket and the first detector assembly 121, which is driven by the detector driving apparatus to move in a direction parallel to the height direction of the wall stand 122 in the plane held by the rotating bracket, and the first detector assembly 121 can further be rotated relative to the support arm to form a certain angle with the wall stand. The first detector assembly 121 has a plate-like structure whose orientation is variable so that the X-ray incident surface can become vertical or horizontal according to the incident direction of the X-rays.

A second detector assembly 131 is included on the examination table apparatus 130, and the selection or use of the first detector assembly 121 and the second detector assembly 131 may be determined on the basis of an image capture site of a patient and/or an image capture protocol, or may be determined on the basis of the position of the object under examination that is obtained by the capturing of a camera, so as to perform image capture and examination at a lying or standing position. FIG. 1 merely shows one example diagram of a wall stand and an examination table, and it should be understood by those skilled in the art that wall stands and/or examination tables of any form or arrangement can be selected, or only the wall stand can be mounted, and the wall stand and/or examination table is not intended to limit the overall scheme of the present invention.

The X-ray imaging system further includes a control apparatus (not shown in the figure), which may be a main control apparatus that is located in a control room, a tube control apparatus that is mounted on the suspension apparatus, a mobile or portable control apparatus, or any combination of the above. The control apparatus may include a source control apparatus and a detector control apparatus. The source control apparatus is used to command the X-ray source to emit X-rays for image exposure. The detector control apparatus is used to select an appropriate detector from among a plurality of detectors and to coordinate the control of various detector functions, such as automatically selecting a corresponding detector according to the position or pose of the object under examination. Alternatively, the detector control apparatus may perform various signal processing and filtering functions, specifically, for initial adjustment of a dynamic range, interleaving of digital image data, etc. In some embodiments, the control apparatus may provide power and timing signals for controlling the operation of the X-ray source and the detector.

In some embodiments, the control apparatus may further be configured to use a digitized signal to reconstruct one or more required images and/or determine useful diagnostic information corresponding to the patient, wherein the control apparatus may include one or more dedicated processors, graphics processing units, digital signal processors, microcomputers, microcontrol apparatuses, application-specific integrated circuits (ASICs), field-programmable gate arrays (FPGAs), or other appropriate processing apparatuses.

Of course, the X-ray imaging system may further include other numbers or configurations or forms of control apparatuses, for example, the control apparatus may be local (e.g., co-located with one or more X-ray imaging systems 100, e.g., within the same facility and/or the same local network); in other implementations, the control apparatus may be remote and thus accessible only via a remote connection (e.g., via the Internet or other available remote access technologies). In a specific implementation, the control apparatus may also be configured in a cloud-like means, and may be accessed and/or used in a means that is substantially similar to the means by which other cloud-based systems are accessed and used.

The X-ray imaging system 100 further includes a storage apparatus (not shown in the figure), and the control apparatus may store digitized signals in the storage apparatus. For example, the storage apparatus may include a hard disk drive, a floppy disk drive, a CD-read/write (CD-R/W) drive, a digital versatile disc (DVD) drive, a flash drive, and/or a solid-state storage apparatus. The storage apparatus is used to store a program that can be executed by a computer. Of course, the storage apparatus may also be integrated together with the control apparatus to effectively use the footprint and/or meet expected imaging requirements.

In one embodiment, the X-ray imaging system 100 further includes an operator workstation. The operator workstation allows a user to receive and evaluate the reconstructed image, and input a control instruction (an operation signal or a control signal). The operator workstation may include a user interface (or a user input device) having a certain form of operator interface, such as a keyboard, a mouse, a voice activated control apparatus, or any other suitable input device, wherein an operator may input an operation signal/control signal to the control apparatus by means of the user interface.

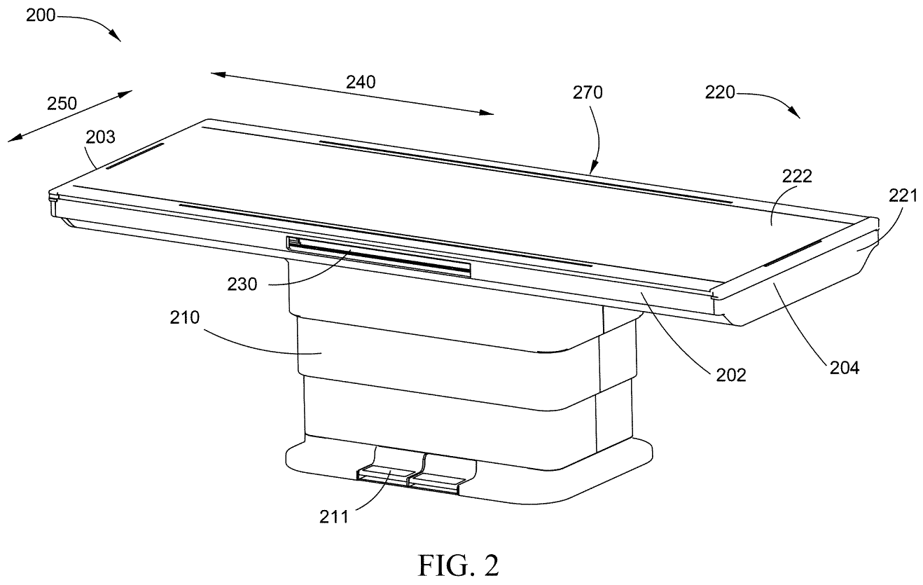

FIG. 2 is a schematic diagram of an examination table 200 according to some embodiments of the present invention. As shown in FIG. 2, the examination table 200 includes a base 210, a table panel assembly 220, and a detector assembly 230. For ease of description, a direction parallel to a long side of a table panel is referred to as a longitudinal direction 240, and a direction parallel to a short side of the table panel is referred to as a transverse direction 250.

In some embodiments, the base 210 has a generally rectangular-shaped, three-dimensional box-like structure, and the base 210 includes a plurality of housings that are sequentially sleeved. The plurality of housings may be sleeved, sequentially from bottom to top, in the housing located thereabove, thereby achieving telescoping so as to adjust the height of the examination table. Specifically, the base 210 includes a pedal 211, and the pedal 211 can be used to control the height of the examination table, and the user can adjust the height of the examination table by means of controlling the pedal 211. It should be understood by those skilled in the art that the base may also be provided in any other form, for example, the base is provided so that the height cannot be adjusted or the height is adjusted in other forms, and the base is not limited to the above description.

The table panel assembly 220 is mounted on the base 210. The table panel assembly 220 is fixed on the base, and is not floating. Moreover, the table panel assembly 220 cannot slide relative to the base 210. Unlike previous floating table panel assemblies, the fixed table panel assembly enables the movement space or mounting/maintenance space required by the examination table to be smaller.

The table panel assembly 220 includes a support frame 221 and a table panel 222. The support frame 221 is mounted on the base 210. The table panel 222 is mounted on the support frame 221, and the table panel 222 is movably connected to the support frame 221. A rotating shaft 270 is formed between the table panel 222 and the support frame 221, and an accommodating space is formed between the table panel 222 and the support frame 221.

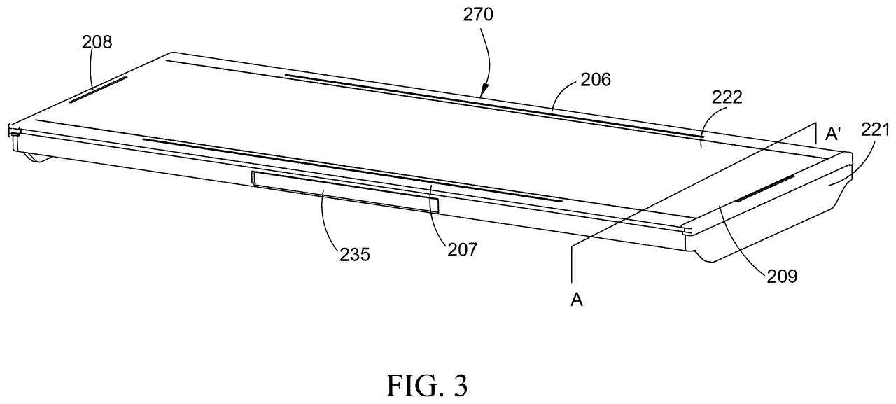

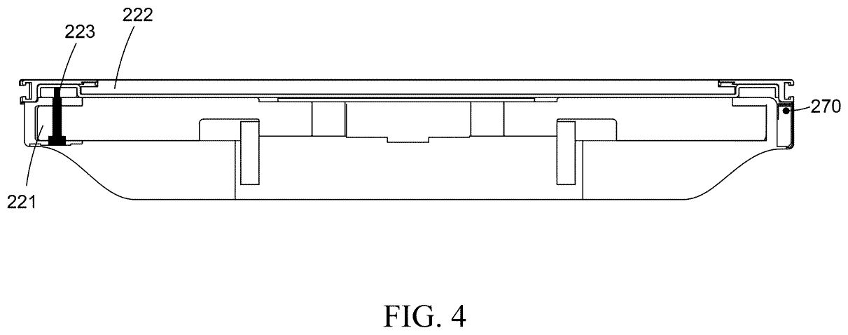

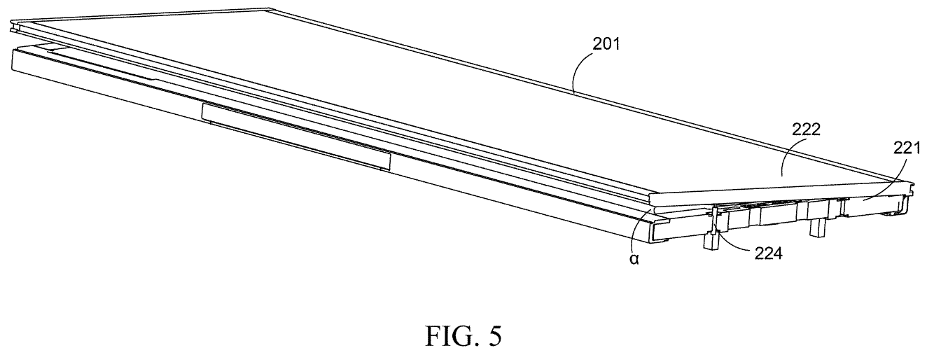

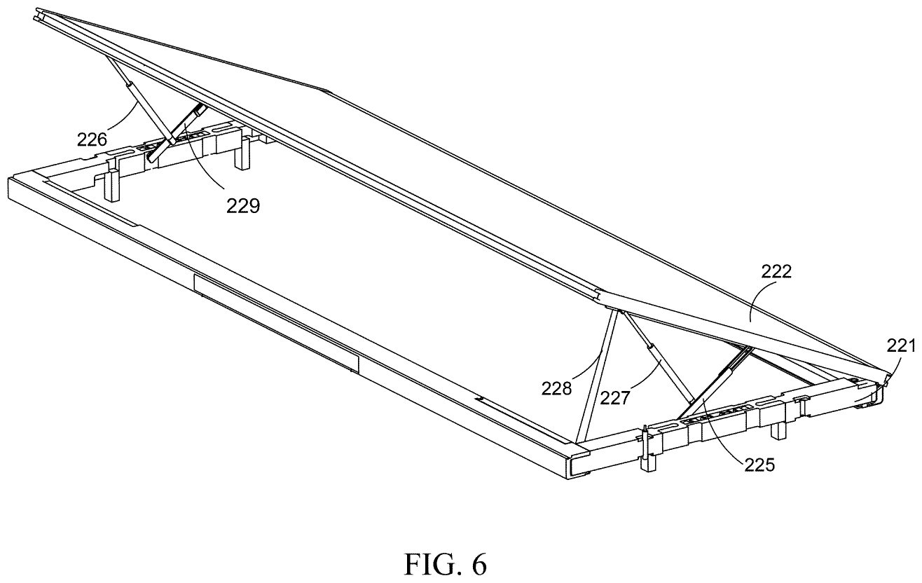

FIG. 3 is a schematic diagram of a table panel assembly in a first state according to some embodiments of the present invention, FIG. 4 is a cross-sectional view of the table panel assembly shown according to FIG. 3 along A-A′, FIG. 5 is a schematic diagram of the table panel assembly in a second state according to some embodiments of the present invention, and FIG. 6 is a schematic diagram of the table panel assembly in a third state according to some embodiments of the present invention. As shown in FIGS. 3 to 6, the table panel assembly 220 includes a support frame 221 and a table panel 222. The table panel 222 is mounted on the support frame 221. The table panel 222 is movably connected to the support frame 221. A rotating shaft 270 is formed between the table panel 222 and the support frame 221. The table panel 222 can be flipped outward relative to the rotating shaft 270. An accommodating space is formed between the table panel 222 and the support frame 221, and the accommodating space can be used for placing and moving a detector.

Specifically, the support frame 221 consists of four frames, internally forming an accommodating space. Specifically, the support frame 221 includes a first frame 201 that forms the rotating shaft, a second frame 202 opposite to the first frame 201, and a third frame 203 and a fourth frame 204 adjacent to the first frame 201, and the four frames are connected together by means of riveting, welding, or the like. Of course, the support frame 221 may also be integrally molded. Specifically, the material of the support frame 221 is aluminum. Of course, the support frame may also be made of other metal materials.

The table panel 222 is a rectangular flat plate structure. The table panel 222 includes a first side 206 near the rotating shaft 270, a second side 207 opposite to the rotating shaft 270, and a third side 208 and a fourth side 209 adjacent to the rotating shaft 270. The table panel 222 may be made using a material having a lower X-ray attenuation, such as being composed of a carbon fiber composite material. The table panel 222 may include a one layer plate or a multilayer plate structure. For example, when the table panel 222 includes the multilayer plate structure, each layer uses a particular material, for example, an inner layer is made of foam and an outer layer is made of a material such as a carbon fiber composite material. The material has an appropriate strength so as to provide stable support for a scanned object. For details, reference may be made to the prior art, which will not be described again here.

In some non-limiting embodiments, the upper surface of table panel 222 is flat. Specifically, the table panel 222 includes a support plate and a pair of side frames extending along the length direction of the support plate, the side frames are used for supporting and fixing the support plate, and the upper surface of the support plate is flush with the upper surfaces of the side frames.

Specifically, the periphery of the table panel 222 is substantially flush with the periphery of the support frame 221. That is, the outer side of the first frame 201 is flush with the first side 206 of the table panel, the outer side of the second frame 202 is flush with the second side 207 of the table panel, the outer side of the third frame 203 is flush with the third side 208 of the table panel, and the outer side of the fourth frame 204 is flush with the fourth side 209 of the table panel. In other words, the perimeter of the table panel is generally equal to the perimeter of the support frame. The term generally refers to being within an error of a value or range that can be expected by those of ordinary skill.

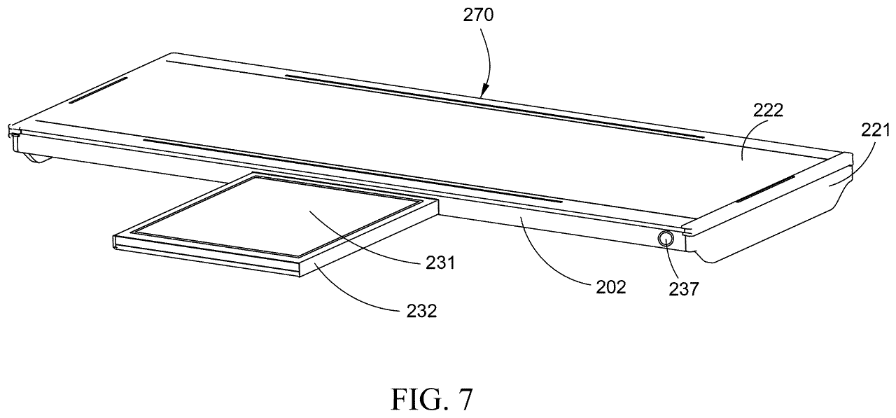

A side edge of the support frame 221 includes an opening 235. Specifically, the side frame of the support frame 221 opposite to the rotating shaft, i.e., the second frame 202, includes an opening 235. The opening 235 can be used for moving a detector assembly 230 in or out. The detector assembly 230 is located in the accommodating space, and can move relative to at least one direction among the transverse and longitudinal directions of the table panel assembly 220. Details will be further described with reference to FIG. 7.

In some non-limiting embodiments, the rotating shaft 270 of the support frame 221 and the table panel 222 is arranged along the length direction of the table panel. That is to say, the table panel can be flipped outward along the length direction, that is, one long side of the table panel is fixed, and the other long side can be lifted or lowered. However, it should be understood by those skilled in the art that the rotating shaft may also be arranged in the short side direction of the table panel, that is, the table panel is flipped outward along the width direction.

The table panel assembly 200 includes a locking unit 223, and the locking unit 223 is used for fixing the second side 207 of the table panel on the support frame 221.

Specifically, the locking unit 223 includes at least two locking blocks, which are located at both ends of the second side of the table panel 222, i.e., two ends of the second frame of the support frame, for fixing the table panel 222 and the support frame 221 together. Specifically, the second frame of the support frame 221 includes a through hole, the locking unit 223 is a screw, and the screw may penetrate the through hole from the bottom of the support frame to be fixed to the bottom of the table panel 222. In some non-limiting embodiments, the locking unit may also include any other locking mode, for example, locking the table panel 222 on the support frame by means of a lock.

In some embodiments, a damper 224 is mounted on each of the third side and the fourth side on the support frame 221, and when the locking unit 223 is opened, the dampers 224 enable the table panel to be able to be automatically flipped outward by a preset angle. Specifically, the dampers 224 are hydraulic dampers.

The third frame and the fourth frame of the support frame, i.e., the frames adjacent to the rotating shaft, are each provided with a damper 224. When the locking unit 223 is opened, for example, the screw is moved out of the through hole, and the table panel 222 can be automatically flipped outward by a preset angle α under the action of the dampers. Specifically, the table panel 222 can change from the first state as shown in FIG. 4 to the second state as shown in FIG. 6. By means of providing the dampers, when the table panel needs to be repaired or opened, the table panel is automatically lifted by means of opening the locking unit, which can enable a user or maintenance personnel to easily lift the table panel, which is convenient for operation, and it is not necessary to utilize other tools to lift the table panel.

In some embodiments, the table panel assembly 220 further includes at least one connecting rod 229/225. The at least one connecting rod is arranged between the table panel 222 and the support frame 221 for connecting the table panel 222 and the support frame 221 when the table panel 222 is lifted. Specifically, the table panel assembly 220 includes a first connecting rod 229 and a second connecting rod 225, the first connecting rod 229 is arranged between the third side 208 of the table panel 222 and the support frame 221, and the second connecting rod 225 is arranged between the fourth side 209 of the table panel 222 and the support frame 221.

The inner side of the table panel 222 includes a sliding slot. One end of the connecting rod is fixed to the inner side of the support frame 221, and the other end is connected within the sliding slot. When the table panel 222 is lifted, the other end of the connecting rod can move within the sliding slot.

Specifically, the inner side of the third side 208 of the table panel 222 includes a first sliding slot. One end of the first connecting rod 229 is connected to the inner side of the third frame of the support frame, and the other end of the first connecting rod 229 is connected within the first sliding slot. The inner side of the fourth side 209 of the table panel 222 includes a second sliding slot. One end of the second connecting rod 225 is connected to the inner side of the fourth frame of the support frame, and the other end of the second connecting rod 225 is connected within the second sliding slot. When the table panel is lifted, the other end of the first connecting rod can slide within the first sliding slot from one end near the rotating shaft to the other end away from the rotating shaft, and at the same time, the other end of the second connecting rod can slide within the second sliding slot from one end near the rotating shaft to the other end away from the rotating shaft, so that the table panel can be lifted.

In another embodiment, the first connecting rod 229 and the second connecting rod 225 are provided as length-adjustable rods. Specifically, the first connecting rod 229 includes a first part and a second part which are movably connected, and the first part and the second part can move relative to each other to change the length of the first connecting rod. A first end of the first connecting rod is connected to the support frame, a second end of the first connecting rod is connected to the table panel, and the first end and the second end of the first connecting rod can rotate relative to the support frame and the table panel. Similarly, the second connecting rod 225 includes a first part and a second part which are movably connected, the first part and the second part can move relative to each other to change the length of the second connecting rod 225, and a first end and a second end of the second connecting rod can rotate relative to the support frame and the table panel. Therefore, when the table panel is lifted, the ends of the first connecting rod and the second connecting rod rotate relative to the support frame and the table panel, and at the same time, the second parts of the first connecting rod and the second connecting rod move relative to the first parts to change the lengths of the first connecting rod and the second connecting rod, thereby lifting the table panel.

In some embodiments, the table panel assembly 220 further includes at least one gas spring 226/227 having one end fixed on the table panel 222 and the other end fixed on at least one connecting rod. Specifically, the table panel assembly 220 further includes a first gas spring 226 and a second gas spring 227. One end of the first gas spring 226 is fixed on the table panel 222, and the other end is fixed on the first connecting rod 229. One end of the second gas spring 227 is fixed on the table panel 222, and the other end is fixed on the second connecting rod 225.

Specifically, one end of the first gas spring 226 is fixed to the inner side of the third side 208 of the table panel, and the other end is fixed on the first connecting rod 229. When the table panel is lifted to a preset position, i.e., when reaching the third state as shown in FIG. 7, a triangle is formed between the first gas spring 226, the first connecting rod 229, and the third side of the table panel. One end of the second gas spring 227 is fixed to the inner side of the fourth side 209 of the table panel, and the other end is fixed on the second connecting rod 225. When the table panel is lifted to a preset position, i.e., when reaching the third state as shown in FIG. 7, a triangle is formed between the second gas spring 227, the second connecting rod 225, and the fourth side of the table panel.

In some embodiments, the gas spring can be used to adjust or change the flipping force required for the table panel during a flipping or lifting process, and the flipping of the table panel is manually controlled. The flipping of the table panel requires manual execution of the user, and the flipping force may refer to an upward impact force required for the table panel during the lifting process and/or a downward pressing force required when the table panel is put down. By means of arranging the gas spring between the table panel and the connecting unit, the gas spring can provide assistance to the user during the lifting or lowering process of the table panel, reducing the flipping force required by the user.

Specifically, the height at which the table panel is lifted is decided by the length of the connecting rod or the length of the sliding slot. When the table panel is lifted to a preset height, due to the limitation of the connecting rod, the table panel cannot continue to be lifted, and the height of the table panel can be fixed due to the action of the gas spring, so that the table panel cannot be automatically lowered and can only be pressed down by relying on external force.

In some embodiments, the table panel assembly 220 further includes at least one support rod 228 for being fixed between the table panel 222 and the support frame 221 when the table panel 222 is lifted to a preset position.

Specifically, when the table panel is in the first state or the second state, that is, the table panel is fixed to the support frame, or the table panel is lifted by a preset angle, the support rod 228 is placed on the inner side of the support frame, for example, is placed within the second frame of the support frame. When the table panel is completely lifted, the user can pick up the support rod 228 and place same within the table panel and the support frame for further fixing the table panel.

Of course, the table panel assembly 220 may include one support rod, or may also include two support rods. The two support rods can be placed at both ends of the second frame, respectively.

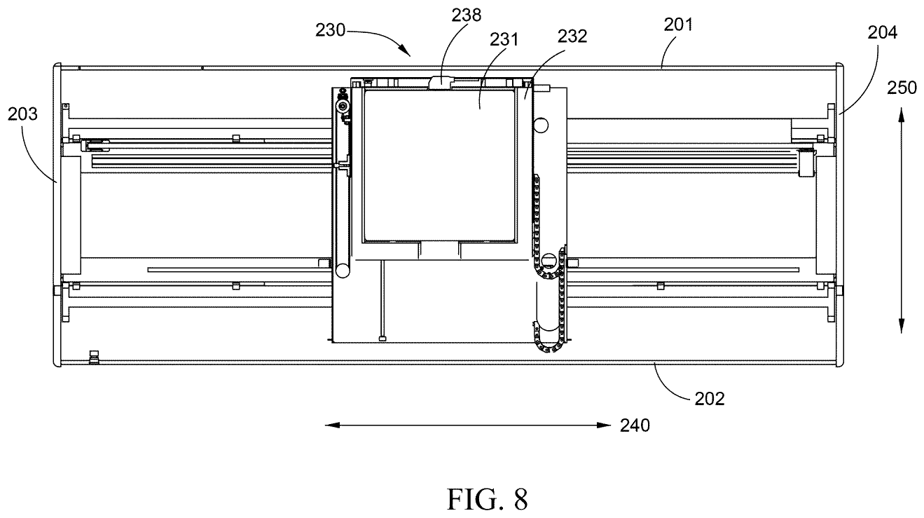

FIG. 7 shows a schematic diagram of a table panel assembly according to some embodiments of the present invention, and FIG. 8 shows a schematic diagram of a detector assembly of an examination table according to some embodiments of the present invention. FIG. 8 shows a perspective view of the examination table with the table panel assembly removed, wherein the table panel assembly is at a middle position with respect to a base.

As shown in FIGS. 7 and 8, the detector assembly 230 is arranged at an upper position of the base 210 so as to be opposite to the rear surface of the table panel. The detector assembly 230 includes a detector panel 231 and a tray 232. The detector panel 231 has an X-ray receiving surface that can be used to receive X-rays, and the detector panel 231 is mounted or fixed on the tray 232.

In some embodiments, the detector assembly 230 includes a first group of moving assemblies for driving the tray 232 and the detector panel 231 to move along a transverse direction 250. Specifically, the detector assembly 230 can move from a side of a first frame 201 to a side of a second frame 202, and therefore, the receiving range of the detector panel 231 can be moved along the transverse direction 250 from one side of the table panel to another side.

Specifically, the first group of moving assemblies includes a first group of synchronous belts, a first group of guide rails, and a first motor. The first group of guide rails is arranged along the transverse direction 250. The bottom of the tray 232 is provided with guide rail slots opposite to the first group of guide rails, so that the tray 232 can move relative to the first group of guide rails. One end of the first group of synchronous belts is fixed on the tray 232, and the other end is connected to the first motor, so as to control the first group of synchronous belts by means of the first motor to drive the tray to move, so that the tray and the detector move along the transverse direction 250. Specifically, the first group of moving assemblies further includes at least one first group of sensors to feed back the position of the tray in the transverse direction.

Specifically, the detector assembly 230 further includes a second group of moving assemblies for driving the tray 232 and the detector panel 231 to move along a longitudinal direction 240. Specifically, the detector assembly 230 can be moved from a side of a third frame 203 to a side of a fourth frame 204, and therefore, the receiving range of the detector panel 231 can be moved along the longitudinal direction from one side of the table panel to another side.

Specifically, the second group of moving assemblies includes a second group of synchronous belts, a second group of guide rails, and a second motor. The second group of guide rails is arranged along the longitudinal direction 240, the first group of guide rails is arranged on the second group of guide rails, and the first group of guide rails and the second group of guide rails are vertically arranged. The tray 232 can move relative to the second group of guide rails. One end of the second group of synchronous belts is fixed on the tray 232, and the other end is connected to the second motor, so as to control the second group of synchronous belts by means of the second motor to drive the tray to move, so as to enable the tray and the detector to move along the longitudinal direction 240. Specifically, the second group of moving assemblies further includes at least one second group of sensors to feed back the position of the tray in the longitudinal direction.

The detector assembly further includes a control module arranged within the base 210. The control module can be connected to a control apparatus and/or an operator console of an X-ray imaging system, and can control the movement of the detector and/or the height adjustment of the examination table for controlling the detector to move in the transverse direction and/or the longitudinal direction. The control module controls the table panel and/or the detector on the basis of input signals from the pedal and/or input signals from the plurality of sensors.

Specifically, a side edge (of the second frame 202) of the support frame 221 is provided with an opening 235 that has a size generally equal to the size of a side edge of the tray, and the opening 235 can allow the tray 232 to carry the detector panel 231 to move into or out of the examination table. Specifically, the tray is provided with a handle, and when the tray is moved out of the examination table by a preset distance, the handle can be exposed outside of the examination table to facilitate the user to operate the tray and the detector panel.

Specifically, the support frame 221 is provided with a tray control button 237. The tray control button 237 can be connected to the tray 232, and controls the tray to move out of the opening 235 by a preset distance. Specifically, it is assumed that the detector panel is located at an edge position of the table panel close to the third side. When the tray control button 237 is pressed, a control signal is sent to the control module, so that the tray is driven by means of the second group of moving assemblies to move along the longitudinal direction 240 to an initial position aligned with the direction of the opening, and then the tray is moved along the transverse direction to an exit position by means of the first group of moving assemblies, wherein the initial position is a position at which the tray is aligned with the direction of the opening, that is, that is, the line connecting the center of the opening and the center of the detector panel is parallel to the transverse direction 250, and the exit position means that at least a part of the tray is moved out of the opening 235.

In some non-limiting embodiments, the tray and the support frame are provided with position-limiting blocks, which can limit the maximum movement distance of the tray when it is moved out of the accommodating space or opening, so that the tray can be moved out to fully expose the detector panel while fixing the position of the tray.

In the process of moving the tray in, by means of moving at least part of the tray into the opening, i.e., entering the accommodating space, the sensors arranged within the accommodating space or within the base can detect the position of the detector panel, and the position is fed back to the control module. The control module can control the movement of the tray at at least one position in the transverse direction and/or the longitudinal direction on the basis of a current position, a target position, and the like.

Therefore, the receiving range of the detector panel 231 can generally cover the entire panel range of the table panel, that is, when the object under examination is lying on the examination table, the detector panel 231 can correspondingly move along the transverse direction and the longitudinal direction to cover the entire region range of the object under examination.

In some non-limiting embodiments, for an application scenario in which image stitching needs to be performed, for example, an entire leg needs to be captured to acquire an X-ray image of the entire leg, the detector is controlled by means of the control module to reach the base of the thigh, and then the detector is moved along the transverse and/or longitudinal direction by means of the first group of moving assemblies and the second group of moving assemblies, so that the receiving surface of the detector can cover the entire leg to acquire the X-ray image.

Specifically, it is possible to cause the detector to reach a first longitudinal position and move along the transverse direction 250 from a first side (a first frame position 201) to a second side (a second frame position 202), and then cause the detector to reach a second longitudinal position and then move along the transverse direction 250 from the second side (the second frame position 202) to the first side (the first frame position 201) to reach a preset longitudinal position. Of course, it is also possible to cause the detector to reach a first transverse position, move along the longitudinal direction 240 from a third side (a third frame position 203) to a fourth side (a fourth frame position 204), then reach a second transverse position, and move along the longitudinal direction from the fourth side (the fourth frame position 204) to the third side (the third frame position 203) until an exposure task is completed.

In some non-limiting embodiments, when the table panel needs to be opened, the locking unit is first opened, so that a side of the table panel is lifted by a preset height or angle under the action of the dampers. The foregoing height can generally enable the user to easily operate the table panel by hand or by the use of other tools. Next, the user can continue to lift the table panel to a preset height. This height is decided by the length of the connecting rod or the length of the sliding slot. Optionally, after the predetermined height is reached, the user can place the support rod between the lifted table panel and the support frame. When the table panel needs to be closed, the support rod is correspondingly removed, then the table panel is pressed down, and finally, the locking unit is fixed to the table panel. The entire process is easy to operate, the process is simplified, the space required is small, and the efficiency is high.

In the table panel assembly and the examination table according to some embodiments of the present invention, the table panel is movably connected to the support frame, enabling the table panel to be flipped outward relative to the support frame, so that the mounting space or maintenance space required by the table panel and/or the examination table is smaller, for example, a space of approximately 1 meter is saved in the length direction of the table panel. Second, the table panel is fixed to the support frame, and the table panel cannot slide relative to the support frame, so that the accommodating space within the support frame can become a moving space of the detector to enable the detector to be moved in both the transverse and longitudinal directions, enabling the receiving range of the detector to generally cover the entire range of the table panel.

The exemplary embodiments of the present invention provide a table panel assembly. The table panel assembly is arranged on a base of an examination table and is used for bearing an object under examination. The table panel assembly includes a support frame and a table panel. The support frame is mounted on the base. The table panel is mounted on the support frame. The table panel is movably connected to the support frame. A rotating shaft is formed between the table panel and the support frame. The table panel can be flipped outward relative to the rotating shaft. An accommodating space is formed between the table panel and the support frame, and the accommodating space can be used for placing and moving a detector.

Specifically, the table panel includes a first side having the rotating shaft, a second side opposite to the rotating shaft, and a third side and a fourth side adjacent to the rotating shaft, and the table panel assembly includes a locking unit for fixing the second side of the table panel on the support frame.

Specifically, a damper is mounted on each of the third side and the fourth side on the support frame, and when the locking unit is opened, the dampers enable the table panel to be able to be automatically flipped outward by a preset angle.

Specifically, the table panel assembly further includes at least one connecting rod arranged between the table panel and the support frame for connecting the table panel and the support frame when the table panel is lifted.

Specifically, an inner side of the table panel includes a sliding slot, one end of the at least one connecting rod is fixed to an inner side of the support frame, the other end is connected within the sliding slot, and when the table panel is lifted, the other end of the connecting rod can move within the sliding slot.

Specifically, the table panel assembly further includes at least one gas spring having one end fixed on the table panel, and the other end fixed on the at least one connecting rod.

Specifically, when the table panel is lifted to a preset position, a triangle is formed between the gas spring, the connecting unit, and the table panel.

Specifically, the table panel assembly further includes: at least one support rod for further fixing between the table panel and the support frame when the table panel is lifted to the preset position.

Specifically, the accommodating space can be used for moving the detector in at least one of transverse and longitudinal directions.

The exemplary embodiments of the present invention further provide an examination table. The examination table includes a base, a support frame, a table panel, and a detector assembly. The support frame is mounted on the base. The table panel is mounted on the support frame. The table panel is movably connected to the support frame. A rotating shaft is formed between the table panel and the support frame, and an accommodating space is formed between the table panel and the support frame. The detector assembly is mounted within the accommodating space, and the detector assembly can move relative to at least one direction among transverse and longitudinal directions of the table panel.

Specifically, a side of the support frame opposite to the rotating shaft includes an opening that can be used to move the detector assembly in or out.

Specifically, the range of movement of the detector assembly is generally the range of the table panel. Specifically, the range of movement of the detector assembly is a range formed by the surface area of the table panel minus the surface area of the support frame.

The detector assembly includes a detector panel, a tray, a first group of moving assemblies, and a second group of moving assemblies. The detector panel is used for receiving X-rays, the tray is used for placing the detector panel, the first group of moving assemblies is used for driving the tray to move along the transverse direction of the table panel, and the second group of moving assemblies is used for driving the tray to move along the longitudinal direction of the table panel.

The detector assembly further includes a charging unit arranged on the tray for charging the detector panel.

Specifically, the table panel includes a first side having the rotating shaft, a second side opposite to the rotating shaft, and a third side and a fourth side adjacent to the rotating shaft, and the table panel assembly includes a locking unit for fixing the second side of the table panel on the support frame.

Specifically, a damper is mounted on each of the third side and the fourth side on the support frame, and when the locking unit is opened, the dampers enable the table panel to be able to be automatically flipped outward by a preset angle.

Specifically, the table panel assembly further includes at least one connecting rod arranged between the table panel and the support frame for connecting the table panel and the support frame when the table panel is lifted.

Specifically, an inner side of the table panel includes a sliding slot, one end of the at least one connecting rod is fixed to an inner side of the support frame, the other end is connected within the sliding slot, and when the table panel is lifted, the other end of the connecting rod can move within the sliding slot.

Specifically, the table panel assembly further includes at least one gas spring having one end fixed on the table panel, and the other end fixed on the at least one connecting rod.

Specifically, when the table panel is lifted to a preset position, a triangle is formed between the gas spring, the connecting unit, and the table panel.

Specifically, the table panel assembly further includes: at least one support rod for further fixing between the table panel and the support frame when the table panel is lifted to the preset position.

The exemplary embodiments of the present invention further provide an X-ray imaging system. The X-ray imaging system includes an examination table. The examination table includes a base, a support frame, a table panel, and a detector assembly. The support frame is mounted on the base. The table panel is mounted on the support frame. The table panel is movably connected to the support frame. A rotating shaft is formed between the table panel and the support frame, and an accommodating space is formed between the table panel and the support frame. The detector assembly is mounted within the accommodating space, and the detector assembly can move relative to at least one direction among transverse and longitudinal directions of the table panel.

Specifically, a side of the support frame opposite to the rotating shaft includes an opening that can be used to move the detector assembly in or out.

Specifically, the range of movement of the detector assembly is generally the range of the table panel. Specifically, the range of movement of the detector assembly is a range formed by the surface area of the table panel minus the surface area of the support frame.

The detector assembly includes a detector panel, a tray, a first group of moving assemblies, and a second group of moving assemblies. The detector panel is used for receiving X-rays, the tray is used for placing the detector panel, the first group of moving assemblies is used for driving the tray to move along the transverse direction of the table panel, and the second group of moving assemblies is used for driving the tray to move along the longitudinal direction of the table panel.

The detector assembly further includes a charging unit arranged on the tray for charging the detector panel.

As used herein, the term “computer” May include any processor-based or microprocessor-based system that includes a system using a microcontrol device, a reduced instruction set computer (RISC), an application-specific integrated circuit (ASIC), a logic circuit, and any other circuit or processor capable of performing the functions described herein. The examples above are exemplary only and are not intended to limit the definition and/or meaning of the term “computer” in any way.

Some exemplary embodiments have been described above; however, it should be understood that various modifications may be made. For example, suitable results can be achieved if the described techniques are performed in a different order and/or if components in the described systems, architectures, devices, or circuits are combined in different ways and/or replaced or supplemented by additional components or equivalents thereof. Accordingly, other implementations also fall within the scope of protection of the claims.

1. A table panel assembly, arranged on a base of an examination table and used for bearing an object under examination, the table panel assembly comprising:
a support frame mounted on the base; and
a table panel mounted on the support frame, wherein the table panel is movably connected to the support frame, a rotating shaft is formed between the table panel and the support frame, the table panel is capable of being flipped outward relative to the rotating shaft, an accommodating space is formed between the table panel and the support frame, and the accommodating space is capable of being used for placing and moving a detector.

2. The table panel assembly according to claim 1, wherein the table panel comprises a first side having the rotating shaft, a second side opposite to the rotating shaft, and a third side and a fourth side adjacent to the rotating shaft, and the table panel assembly comprises a locking unit for fixing the second side of the table panel on the support frame.

3. The table panel assembly according to claim 2, wherein a damper is mounted on each of the third side and the fourth side on the support frame, and when the locking unit is opened, the dampers enable the table panel to be capable of being automatically flipped outward by a preset angle.

4. The table panel assembly according to claim 2, further comprising:
at least one connecting rod arranged between the table panel and the support frame for connecting the table panel and the support frame when the table panel is lifted.

5. The table panel assembly according to claim 4, wherein an inner side of the table panel comprises a sliding slot, one end of the at least one connecting rod is fixed to an inner side of the support frame, the other end is connected within the sliding slot, and when the table panel is lifted, the other end of the connecting rod is capable of moving within the sliding slot.

6. The table panel assembly according to claim 4, further comprising:
at least one gas spring having one end fixed on the table panel, and the other end fixed on the at least one connecting rod.

7. The table panel assembly according to claim 6, wherein when the table panel is lifted to a preset position, a triangle is formed between the gas spring, the said at least one connecting rod, and the table panel.

8. The table panel assembly according to claim 4, further comprising: at least one support rod for further fixing between the table panel and the support frame when the table panel is lifted to a preset position.

9. The table panel assembly according to claim 1, wherein the accommodating space is capable of being used for moving the detector in at least one direction among transverse and longitudinal directions.

10. An examination table, comprising:
a base;
a support frame mounted on the base; and
a table panel mounted on the support frame, wherein the table panel is movably connected to the support frame, a rotating shaft is formed between the table panel and a side of the support frame, and an accommodating space is formed between the table panel and the support frame; and
a detector assembly mounted within the accommodating space, wherein the detector assembly is capable of moving relative to at least one direction among transverse and longitudinal directions of the table panel within the accommodating space.

11. The examination table according to claim 10, wherein a side of the support frame opposite to the rotating shaft comprises an opening that is capable of being used to move the detector assembly in or out.

12. The examination table according to claim 10, wherein the detector assembly comprises:
a detector panel for receiving X-rays;
a tray for placing the detector panel;
a first group of moving assemblies for driving the tray to move in the transverse direction of the table panel; and
a second group of moving assemblies for driving the tray to move in the longitudinal direction of the table panel.

13. The examination table according to claim 10, wherein the detector assembly further comprises a charging unit arranged on the tray for charging the detector panel.

14. An X-ray imaging system, comprising the examination table according to claim 10.
